(19) United States
(12) Patent Application Publication
(10) Pub. No.: US 2025/0276984 A1
(43) Pub. Date: Sep. 4, 2025

(54) SOLID STATE FORMS OF HER2 INHIBITORS

(71) Applicant: Iambic Therapeutics, Inc., San Diego, CA (US)

(72) Inventors: Iriny BOTROUS, San Diego, CA (US); Joseph Michael DENNIS, San Diego, CA (US); Svitlana KULYK, San Diego, CA (US); Laurent GOMEZ, San Diego, CA (US); Shawn Eric WRIGHT, San Diego, CA (US); Wallace Deshaughn DERRICOTTE, Atlanta, GA (US); Thomas Francis MILLER, III, La Jolla, CA (US); Frederick Roy MANBY, Bristol (GB); Rampurna Prasad GULLAPALLI, San Diego, CA (US)

(21) Appl. No.: 19/057,838

(22) Filed: Feb. 19, 2025

Related U.S. Application Data
(63) Continuation of application No. PCT/US2025/016500, filed on Feb. 19, 2025.
(60) Provisional application No. 63/555,758, filed on Feb. 20, 2024.

(51) Int. Cl. C07D 519/00; A61K 31/53

(52) U.S. Cl. CPC C07D 519/00; A61K 31/53

(57) ABSTRACT

Provided are solid state forms of (E)-1-(4-(4-((4-([1,2,4]triazolo[1,5-a]pyridin-7-yloxy)-3-methylphenyl)amino)pyrrolo[2,1-f][1,2,4]triazin-5-yl)piperidin-1-yl)-4-(dimethylamino)but-2-en-1-one and salts thereof. Such solid state forms re useful in preparation of pharmaceutical compositions for the treatment of diseases.

### CROSS-REFERENCE

This application is a continuation of International Application No.: PCT/US2025/016500, filed Feb. 19, 2025, which claims the benefit of U.S. Provisional Application No. 63/555,758, filed Feb. 20, 2024, which is hereby incorporated by reference in its entirety herein.

### BACKGROUND

HER2 (also referred to as Her2) belongs to the epidermal growth factor receptor (EGFR) family. This family is composed of four HER receptors: human epidermal growth factor receptor 1 (HER1) (also termed EGFR), HER2, human epidermal growth factor receptor 3 (HER3), and human epidermal growth factor receptor 4 (Her4) Yarden Y, Sliwkowski M X. Untangling the ErbB signaling network. Nature Reviews. Molecular Cell Biology. 2001; 2 (2): 127-137. The HER2 receptor is a 185 kDa transmembrane protein that is encoded by the HER2 (also known as erb-b2 receptor tyrosine kinase 2 [ERBB2]) gene. HER2 is normally expressed on cell membranes of epithelial cells of several organs like the lungs, breast and the skin, as well as gastrointestinal, reproductive, and urinary tract [17]. HER2 in normal cells is expressed at low levels, whereas in HER2-positive cancer cells, there is an increase in the number of HER2 gene copies (gene amplification) and HER2 receptors with up to 40-to-100-fold increase in protein overexpression. The increased amount of cell surface HER2 receptors associated with HER2 overexpression leads to increased receptor-receptor interactions, provoking a sustained tyrosine phosphorylation of the kinase domain and therefore constant activation of the signaling pathways.

Tumors driven by HER2 mutations or HER2 wild type over expression may benefit from tyrosine kinase inhibitors that target HER2.

In many cancer types, tumor cells make extra copies of the gene that produces the HER2 protein, known as gene amplification. The resulting flood of HER2 protein causes cancer cells to grow uncontrollably, and cancer cells may also become dependent on the extra HER2 such that stopping the production of the HER2 protein can cause the cancer cells to stop growing or die. In breast cancer, 15-20% of tumors overexpress HER2 and HER2-targeted treatments are commonly used.

HER2 overexpression has been described in not only breast and gastric/gastroesophageal junction carcinomas, but somatic HER2 mutations have also been described at low frequencies in a variety of human cancers including non-small cell lung cancer, colorectal cancer, and bladder cancer. Breast cancer is a heterogeneous disease comprising various molecular subtypes, with approximately 15-20% of cases characterized by HER2-positive overexpression. Targeted therapies, such as trastuzumab, have demonstrated substantial clinical benefits for these subjects, although challenges persist, including the development of treatment resistance. Given the central role of HER2 expression in driving the disease, combining HER2-directed agents with trastuzumab has gained attention as a strategy to address HER2-related aspects of the disease from multiple angles, offering potential for improved treatment outcomes. Despite recent advances in the treatment of metastatic NSCLC, the absolute number of long-term survivors remains low.

In metastatic CRC, 3% to 5% of subjects present with HER2 alterations, and the prognosis for subjects with metastatic colorectal cancer remains poor with 5-year survival rates of 5% or less. The 5-year relative survival rate for subjects with metastatic bladder cancer is only 8% (NIH, 2023). Bladder cancer ranks third among all cancers in terms of HER2 overexpression, carrying as much as 6% to 17% of gene mutations and/or amplification in tumor tissue samples. HER2 overexpression is associated with pathological malignancy and poor prognosis indicators including carcinoma in situ, multifocal tumor, large tumor size, high tumor stage and grade, lymph node metastasis, progression, recurrence, and papillary tumor.

There is an urgent unmet need for novel anti-HER2 monotherapies and dual therapies designed to treat subjects with HER2-driven cancers, which exhibit robust activity against both HER2 and HER2 mutants.

### SUMMARY

Disclosed herein is a solid form of (E)-1-(4-(4-((4-([1,2,4]triazolo[1,5-a]pyridin-7-yloxy)-3-methylphenyl)amino)pyrrolo[2,1-f][1,2,4]triazin-5-yl)piperidin-1-yl)-3-(dimethylamino)prop-2-en-1-one, shown as Compound 1:

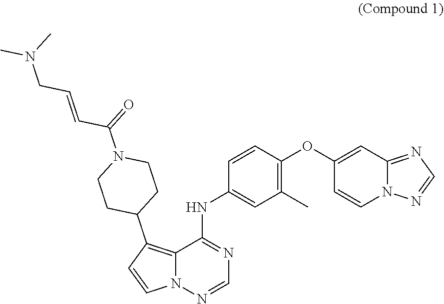 [SMILES: Cc1cc(Nc2ncnn3ccc(C4CCN(C(=O)/C=C/CN(C)C)CC4)c23)ccc1Oc1ccn2ncnc2c1]

wherein the solid form is crystalline.

Disclosed herein is a pharmaceutical composition comprising any one of the solid forms described herein, and one or more pharmaceutically acceptable excipients or carriers.

Disclosed herein is a method of treating cancer in a subject, the method comprising administering to the subject an effective amount any of the solid forms disclosed herein, or any of the pharmaceutical compositions disclosed herein.

### BRIEF DESCRIPTION OF THE DRAWINGS

FIG. 1A and FIG. 1B illustrates the X-ray powder diffraction (XRPD) pattern of Compound 1 Form A.

FIG. 2 illustrates DSC and TGA thermographs of Compound 1 Form A.

FIG. 3 illustrates the cycling DSC thermogram of Compound 1 Form A.

FIG. 4 illustrates the DVS plot of Compound 1 Form A.

FIG. 5A and 5B illustrate shifts in the XRPD diffractograms of Compound 1 Form A under various conditions.

### DETAILED DESCRIPTION

Compound 1 may be used to treat subjects with HER2-driven cancers.

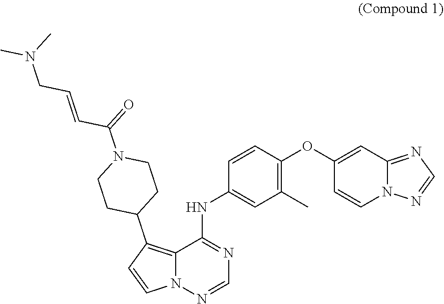 [SMILES: Cc1cc(Nc2ncnn3ccc(C4CCN(C(=O)/C=C/CN(C)C)CC4)c23)ccc1Oc1ccn2ncnc2c1]

### (E)-1-(4-(4-((4-([1,2,4]triazolo[1,5-a]pyridin-7-yloxy)-3-methylphenyl)amino)pyrrolo[2,1-f][1,2,4]triazin-5-yl)piperidin-1-yl)-4-(dimethylamino)but-2-en-1-one

Provided herein are compositions comprising crystalline and amorphous solid state forms of Compound 1.

### Crystalline Compound 1 Form A

The compounds described herein can be in a solid form, wherein the solid form is crystalline. In some embodiments is a solid form of (E)-1-(4-(4-((4-([1,2,4]triazolo[1,5-a]pyridin-7-yloxy)-3-methylphenyl)amino)pyrrolo[2,1-f][1,2,4]triazin-5-yl)piperidin-1-yl)-4-(dimethylamino)but-2-en-1-one, shown as Compound 1:

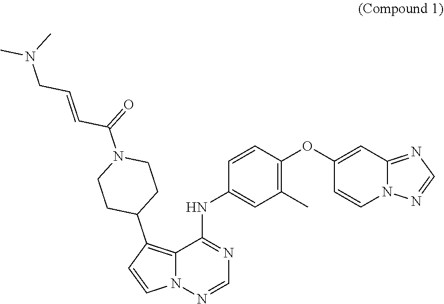 [SMILES: Cc1cc(Nc2ncnn3ccc(C4CCN(C(=O)/C=C/CN(C)C)CC4)c23)ccc1Oc1ccn2ncnc2c1]

wherein the solid form is crystalline.

The solid form can exhibit any X-ray powder diffraction reflection as described herein. In some embodiments, the solid form exhibits an X-ray powder diffraction (XRPD) reflection at a 2-theta value of 15.3°±0.3. In some embodiments, the solid form exhibits an X-ray powder diffraction (XRPD) reflection at a 2-theta value of 20.8°±0.3. In some embodiments, the solid form exhibits an X-ray powder diffraction (XRPD) reflection at a 2-theta value of 19.0°±0.3. In some embodiments, the solid form exhibits an X-ray powder diffraction (XRPD) reflection at a 2-theta value of 12.4°±0.3 and 13.4°±0.3. In some embodiments, the solid form exhibits an X-ray powder diffraction (XRPD) reflection at a 2-theta value of 5.6°±0.3, 6.7°±0.3, and 8.2°±0.3. In some embodiments, the solid form exhibits an X-ray powder diffraction (XRPD) reflection at a 2-theta value of 16.4°±0.3, 16.5°±0.3, and 19.4°±0.3. In some embodiments, the solid form exhibits an X-ray powder diffraction (XRPD) reflection at a 2-theta value of 17.5°±0.3 and 22.8°±0.3.

In some embodiments, the solid form exhibits an X-ray powder diffraction (XRPD) reflection at a 2-theta value of 6.7°±0.3. In some embodiments, the solid form exhibits an X-ray powder diffraction (XRPD) reflection at a 2-theta value of 5.6°±0.3 and 8.2°±0.3. In some embodiments, the solid form exhibits an X-ray powder diffraction (XRPD) reflection at a 2-theta value of 15.3°±0.3, 20.8°±0.3, and 19.0°±0.3. In some embodiments, the solid form exhibits an X-ray powder diffraction (XRPD) reflection at a 2-theta value of 12.4°±0.3 and 13.4°±0.3. In some embodiments, the solid form exhibits an X-ray powder diffraction (XRPD) reflection at a 2-theta value of 17.5°±0.3 and 22.8°±0.3. In some embodiments, the solid form exhibits an X-ray powder diffraction (XRPD) reflection at a 2-theta value of 16.4°±0.3, 16.5°±0.3, and 19.4°±0.3.

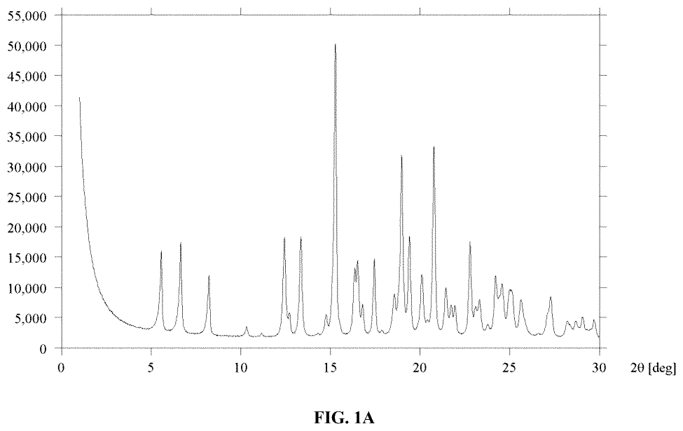

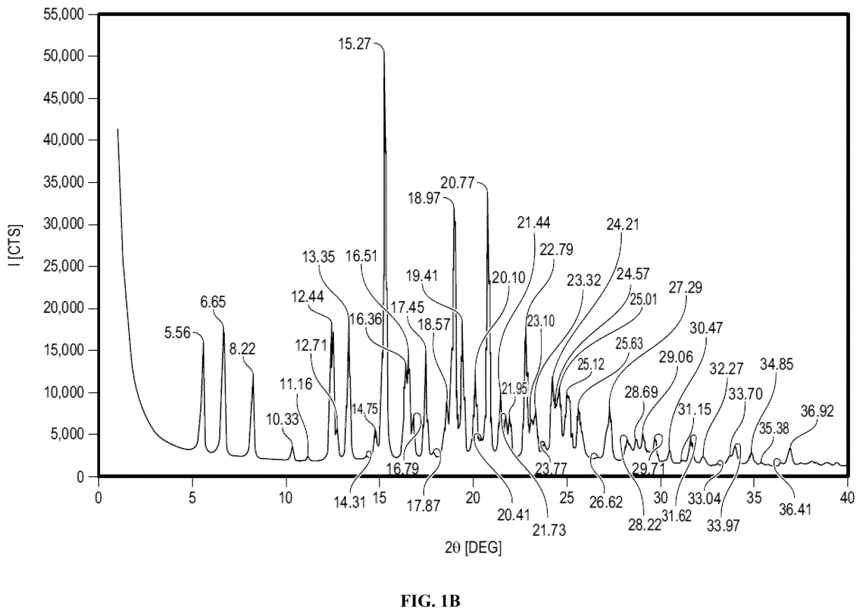

In some embodiments, the solid form exhibits at least one X-ray powder diffraction (XRPD) reflections at a 2-theta value of 5.6°±0.3, 6.7°±0.3, 8.2°±0.3, 12.4°±0.3, 13.4°±0.3, 15.3°±0.3, 16.4°±0.3, 16.5°±0.3, 17.5°±0.3, 19.0°±0.3, 19.4°±0.3, 20.8°±0.3, and 22.8°±0.3. In some embodiments, the solid form exhibits at least two X-ray powder diffraction (XRPD) reflections at a 2-theta value of 5.6°±0.3, 6.7°±0.3, 8.2°±0.3, 12.4°±0.3, 13.4°±0.3, 15.3°±0.3, 16.4°±0.3, 16.5°±0.3, 17.5°±0.3, 19.0°±0.3, 19.4°±0.3, 20.8°±0.3, and 22.8°±0.3. In some embodiments, the solid form exhibits at least three X-ray powder diffraction (XRPD) reflections at a 2-theta value of 5.6°±0.3, 6.7°±0.3, 8.2°±0.3, 12.4°±0.3, 13.4°±0.3, 15.3°±0.3, 16.4°±0.3, 16.5°±0.3, 17.5°±0.3, 19.0°±0.3, 19.4°±0.3, 20.8°±0.3, and 22.8°±0.3. In some embodiments, the solid form exhibits at least four X-ray powder diffraction (XRPD) reflections at a 2-theta value of 5.6°±0.3, 6.7°±0.3, 8.2°±0.3, 12.4°±0.3, 13.4°±0.3, 15.3°±0.3, 16.4°±0.3, 16.5°±0.3, 17.5°±0.3, 19.0°±0.3, 19.4°±0.3, 20.8°±0.3, and 22.8°±0.3. In some embodiments, the solid form exhibits at least five X-ray powder diffraction (XRPD) reflections at a 2-theta value of 5.6°±0.3, 6.7°±0.3, 8.2°±0.3, 12.4°±0.3, 13.4°±0.3, 15.3°±0.3, 16.4°±0.3, 16.5°±0.3, 17.5°±0.3, 19.0°±0.3, 19.4°±0.3, 20.8°±0.3, and 22.8°±0.3. In some embodiments, the solid form exhibits at least six X-ray powder diffraction (XRPD) reflections at a 2-theta value of 5.6°±0.3, 6.7°±0.3, 8.2°±0.3, 12.4°±0.3, 13.4°±0.3, 15.3°±0.3, 16.4°±0.3, 16.5°±0.3, 17.5°±0.3, 19.0°±0.3, 19.4°±0.3, 20.8°±0.3, and 22.8°±0.3. In certain embodiments, the margin of error for any one of the reflections of Compound 1 is selected from ±0.5; ±0.4; ±0.3; ±0.2; ±0.1; and ±0.05. In some embodiments, Compound 1 exhibits the X-ray powder diffraction pattern substantially similar to that shown in FIG. 1A and FIG. 1B. In some embodiments, Compound 1 exhibits at least one of the X-ray powder diffraction pattern reflections in Table 2.

In some embodiments, the solid form exhibits the X-ray powder diffraction (XRPD) pattern as shown in FIG. 1A and FIG. 1B.

In some embodiments, the X-ray powder diffraction (XRPD) was collected using a Cu-Kα radiation source.

In some embodiments, the solid form exhibits a differential scanning calorimetry thermograph comprising an endothermic peak at 171.3±5.0° C. In some embodiments, the solid form exhibits a differential scanning calorimetry thermograph comprising an endothermic onset at 169.7±5.0° C. In certain embodiments, the margin of error for the endotherms of the crystalline solid state of Compound 1 are selected from ±15.0; ±10.0; ±5.0; and ±2.0.

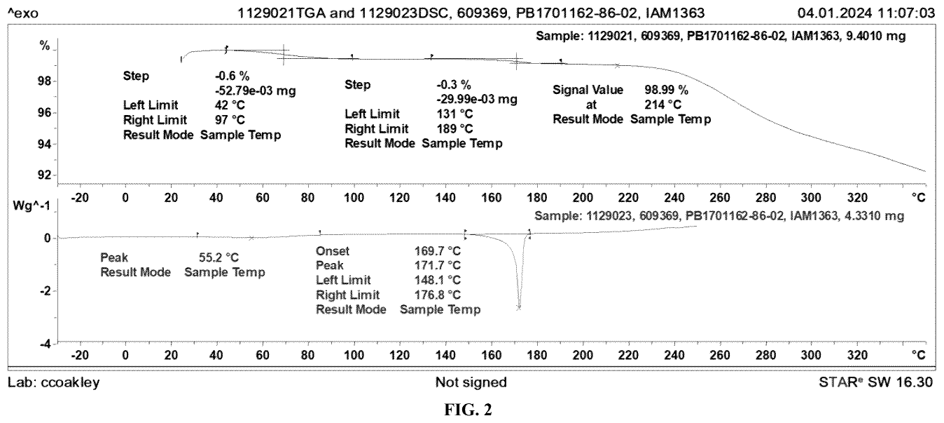

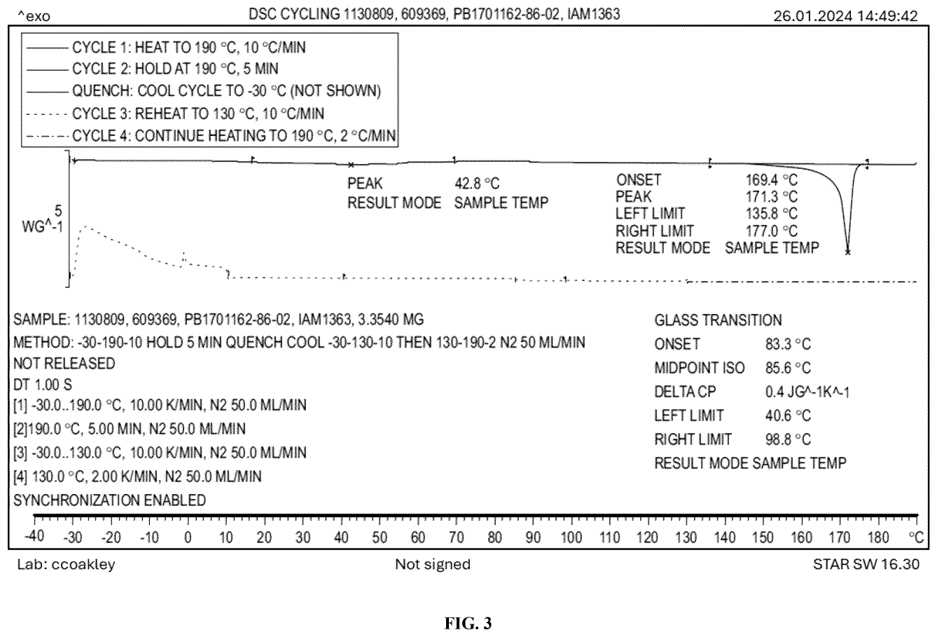

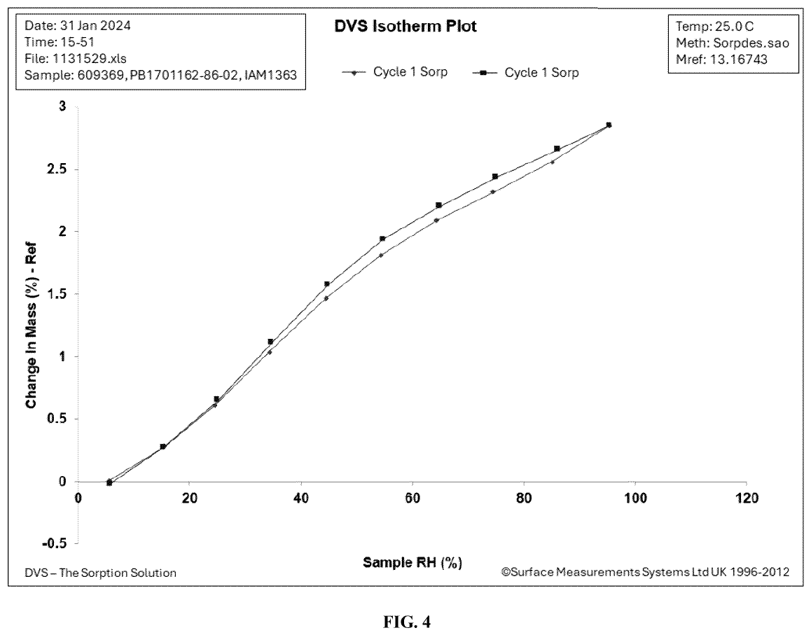

In some embodiments, the solid form exhibits a differential scanning calorimetry (DSC) thermograph as shown in FIG. 2. In some embodiments, the solid form exhibits a cycling DSC thermogram as shown in FIG. 3.

In some embodiments, the solid form exhibits less than 1.0%±0.3 weight loss up to 214° C.±10.0 as determined by thermogravimetric analysis. In some embodiments, the solid form exhibits less than 1.0%±0.3 weight loss up to 190° C.±10.0 as determined by thermogravimetric analysis. In certain embodiments, the margin of error for the TGA weight loss for the crystalline solid state of Compound 1 is selected from ±2.0; ±1.0; ±0.5; ±0.3; and ±0.1.

In some embodiments, the solid form exhibits a TGA thermograph as shown in FIG. 2.

In some embodiments, the solid form is substantially free of other crystalline or amorphous forms. In some embodiments, the amount of other crystalline or amorphous forms is 10% (w/w) or less. In some embodiments, the amount of other crystalline or amorphous forms is 5% (w/w) or less. In some embodiments, the amount of other crystalline or amorphous forms is 4% (w/w) or less. In some embodiments, the amount of other crystalline or amorphous forms is 3% (w/w) or less. In some embodiments, the amount of other crystalline or amorphous forms is 2% (w/w) or less. In some embodiments, the amount of other crystalline or amorphous forms is 1% (w/w) or less.

In some embodiments, the solid form is substantially free of impurities. In some embodiments, the amount of impurities is 5% or less. In some embodiments, the amount of impurities is 4% or less. In some embodiments, the amount of impurities is 3% or less. In some embodiments, the amount of impurities is 2% or less. In some embodiments, the amount of impurities is 1% or less.

In some embodiments is a pharmaceutical composition comprising any one of the solid forms of the crystalline compound described herein, and one or more pharmaceutically acceptable excipients or carriers.

### XRPD Peak Shifts

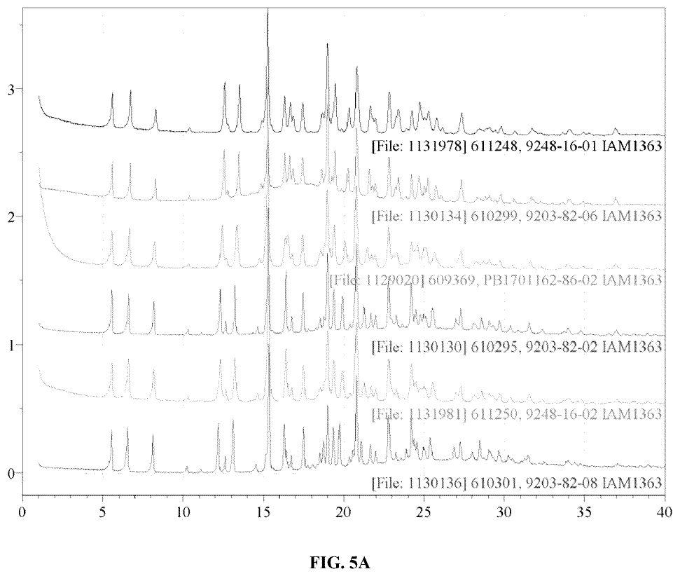

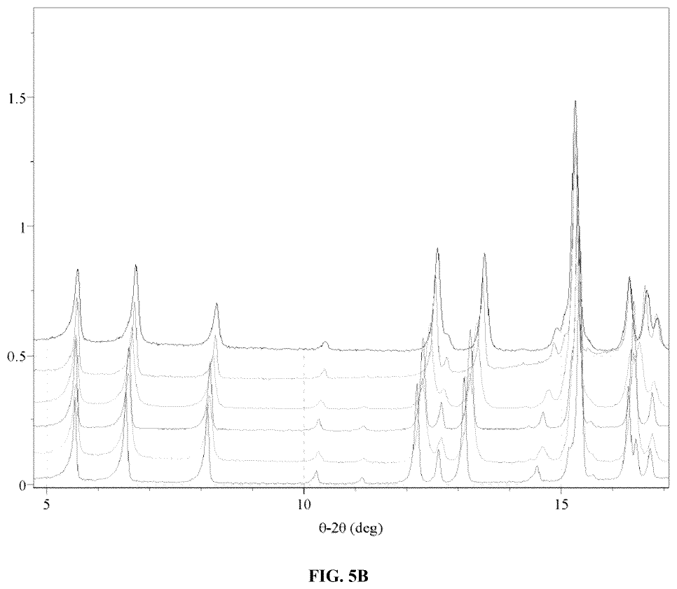

In certain embodiments, the margin of error for any one of the XRPD reflections of Compound 1 is selected from ±0.5; ±0.4; ±0.3; ±0.2; ±0.1; and ±0.05. XRPD patterns of various samples of Compound 1 Form A exhibited peaks shifts of ±0.3 under various conditions. In some embodiments, XRPD patterns of various samples of Compound 1 Form A may exhibit peaks shifts of ±0.5. Illustration of the shifts is provided in FIG. 5A and FIG. 5B. The figures show stacked XRPD diffractograms under the following conditions: stored at 0% relative humidity; slurried in DMAC/MTBE, as obtained from the procedure described in Example 1; slurried in acetonitrile; stored at 75% relative humidity; slurried in 40:60 acetone/water slurry (a_{w} 0.92).

### Amorphous Compound 1

Amorphous form of Compound 1 was generated by dissolving or slurrying Compound 1 in DCM or methanol followed by rapid evaporation of the solvent. The amorphous solid state forms exhibited an absence of interlattice plane intervals as characterized by XRPD. Pharmaceutical Compositions

In certain embodiments, Compound 1 Form A is administered as a pure chemical. In other embodiments, Compound 1 Form A is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

### Methods

Provided herein are methods which use the compounds described herein. The methods can be useful for treating a disease.

In some embodiments is method of treating cancer in a subject, the method comprising administering to the subject an effective amount of the solid form of any one of claims 1 to Error! Reference source not found. [symbol] , or the pharmaceutical composition described herein.

In some embodiments, the cancer is HER2 mediated. In some embodiments, the cancer is brain cancer, colorectal cancer, breast cancer, bladder cancer, or non-small cell lung cancer. In some embodiments, the cancer is brain cancer. In some embodiments, the cancer is colorectal cancer. In some embodiments, the cancer is breast cancer. In some embodiments, the cancer is bladder cancer. In some embodiments, the cancer is non-small cell lung cancer.

In some embodiments, the method further comprises administering an additional therapeutic agent. In some embodiments, the additional therapeutic agent is a monoclonal antibody. In some embodiments, the monoclonal antibody is pertuzumab, margetuximab, or trastuzumab. In some embodiments, the monoclonal antibody is pertuzumab. In some embodiments the monoclonal antibody is margetuximab. In some embodiments, the monoclonal antibody is trastuzumab. In some embodiments, the additional therapeutic agent is a monoclonal antibody-drug conjugate. In some embodiments, wherein the monoclonal antibody-drug conjugate is ado-trastuzumab emtansine or trastuzumab deruxtecan.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables.

“Pharmaceutically acceptable salt” refers to a salt which is acceptable for administration to a patient, such as a mammal (e.g., salts having acceptable mammalian safety for a given dosage regime). Contemplated pharmaceutically acceptable salt forms include, without limitation, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug. Such salts can be derived from pharmaceutically acceptable inorganic or organic bases and from pharmaceutically-acceptable inorganic or organic acids, depending on the particular substituents found on the compounds described herein.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. In another example, a salt can be prepared by reacting the free base and acid in an organic solvent.

When compounds of the present disclosure contain relatively acidic functionalities, base addition salts can be obtained by contacting the neutral form of such compounds with a sufficient amount of the desired base (i.e. a primary, secondary, tertiary, quaternary, or cyclic amine; an alkali metal hydroxide; alkaline earth metal hydroxide; or the like), either neat or in a suitable inert solvent. The desired acid can be, for example, a pyranosidyl acid (such as glucuronic acid or galacturonic acid), an alpha-hydroxy acid (such as citric acid or tartaric acid), an amino acid (such as aspartic acid or glutamic acid), an aromatic acid (such as benzoic acid or cinnamic acid), a sulfonic acid (such as p-toluenesulfonic acid or ethanesulfonic acid), or the like. In some embodiments, salts can be derived from pharmaceutically acceptable acids such as acetic, trifluoroacetic, propionic, ascorbic, benzenesulfonic, benzoic, camphorsulfonic, citric, ethanesulfonic, fumaric, glycolic, gluconic, glucoronic, glutamic, hippuric, hydrobromic, hydrochloric, isethionic, lactic, lactobionic, maleic, malic, malonic, mandelic, oxalic, methanesulfonic, mucic, naphthalenesulfonic, nicotinic, nitric, pamoic, pantothenic, phosphoric, succinic, sulfuric, sulfamic, hydroiodic, carbonic, tartaric, p-toluenesulfonic, pyruvic, aspartic, benzoic, cinnamic, anthranilic, mesylic, salicylic, p-hydroxybenzoic, phenylacetic, embonic (pamoic), ethanesulfonic, benzenesulfonic, 2-hydroxyethanesulfonic, sulfanilic, stearic, cyclohexylsulfamic, cyclohexylaminosulfonic, quinic, algenic, hydroxybutyric, galactaric and galacturonic acid and the like.

Also included are salts of amino acids such as arginate and the like, and salts of organic acids like glucuronic or galactunoric acids and the like (see, for example, Berge, S. M. et al., “Pharmaceutical Salts,” J. Pharmaceutical Science, 1977, 66:1-19). Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

The pharmaceutically acceptable salt of the different compounds may be present as a complex. Examples of complexes include 8-chlorotheophylline complex (analogous to, e.g., dimenhydrinate: diphenhydramine 8-chlorotheophylline (1:1) complex; Dramamine) and various cyclodextrin inclusion complexes.

The term “carrier” is also meant to include microspheres, liposomes, micelles, nanoparticles (naturally-equipped nanocarriers, for example, exosomes), and the like. It is known that exosomes can be highly effective drug carriers, and there are various ways in which drugs can be loaded into exosomes, including those techniques described in J Control Release. 2015 Dec. 10; 219:396-405, the contents of which are incorporated by reference in its entirety.

As used herein, the terms “treat,” “treating,” “therapy,” “therapies,” and like terms refer to the administration of material, e.g., any one or more compound(s) as described herein in an amount effective to inhibit Her2, including wild-type Her2 and mutant Her2 such as Her2 with YVMA insertion mutations. In other embodiments of this disclosure, these terms apply to the administration of the compounds of this disclosure to subjects that have disease states associated with Her2 overexpression and/or HER2 amplification. In other embodiments, the terms “treat,” “treating,” “therapy,” “therapies,” and like terms refer to the administration of material, e.g., any one or more compound(s) as described herein is an amount effective to prevent, alleviate, or ameliorate one or more symptoms of a disease or condition, i.e., indication, and/or to prolong the survival of the subject being treated. In other embodiments of this disclosure, these terms apply to the administration of the compounds of this disclosure to subjects that have disease states associated with Her2 overexpression and/or HER2 amplification.

The terms “prevent,” “preventing,” “prevention” and grammatical variations thereof as used herein, refers to a method of partially or completely delaying or precluding the onset or recurrence of a disease, disorder or condition and/or one or more of its attendant symptoms or barring a subject from acquiring or reacquiring a disorder or condition or reducing a subject's risk of acquiring or requiring a disorder or condition or one or more of its attendant symptoms.

As used herein, the term “subject,” “animal subject,” and the like refers to a living organism including, but not limited to, human and non-human vertebrates, e.g. any mammal, such as a human, other primates, sports animals and animals of commercial interest such as cattle, horses, ovines, or porcines, rodents, or pets such as dogs and cats.

The term “administering” refers to oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

In the present context, the term “therapeutically effective” or “effective amount” indicates that a compound or material or amount of the compound or material when administered is sufficient or effective to prevent, alleviate, or ameliorate one or more symptoms of a disease, disorder or medical condition being treated, and/or to prolong the survival of the subject being treated. The therapeutically effective amount will vary depending on the compound, the disease, disorder or condition and its severity and the age, weight, etc., of the mammal to be treated. In general, satisfactory results in subjects are indicated to be obtained at a daily dosage of from about 0.1 to about 10 g/kg subject body weight. In some embodiments, a daily dose ranges from about 0.10 to 10.0 mg/kg of body weight, from about 1.0 to 3.0 mg/kg of body weight, from about 3 to 10 mg/kg of body weight, from about 3 to 150 mg/kg of body weight, from about 3 to 100 mg/kg of body weight, from about 10 to 100 mg/kg of body weight, from about 10 to 150 mg/kg of body weight, or from about 150 to 1000 mg/kg of body weight. The dosage can be conveniently administered, e.g., in divided doses up to four times a day or in sustained-release form.

As used herein, the term “Her2 mediated disease or condition” (which is also meant to mean “Her2 mediated disease or condition” as well as “wild-type Her2 and/or mutant Her2 mediated disease or condition”) refers to a disease or condition in which the biological function of Her2 affect the development and/or course of the disease or condition, and/or in which modulation of the interaction of Her2 alters the development, course, and/or symptoms. A of Her2 mediated disease or condition includes a disease or condition for which the disruption Her2 interactions (for example, by inhibiting Her2 with YVMA insertion mutations) provides a therapeutic benefit, e.g. wherein treatment with Her2 inhibitors, including compounds described herein, provides a therapeutic benefit to the subject suffering from or at risk of the disease or condition. A Her2 mediated disease or condition is intended to include a cancer or tumor where there is activation of Her2. In another embodiments of this disclosure, Her2 mediated diseases or conditions are associated with Her2 overexpression and/or Her2 amplification. A Her2 mediated disease or condition is also intended to include various human carcinomas, including those of the lung, breast, stomach, ovary, colon, bladder, pancreatic cancer, biliary cancer, endometrial cancer, lung, uterine cervix, head and neck, gastric and esophageal cancer as well as uterine serous endometrial carcinoma, as well any associated comorbidities such as pulmonary disorder, hypertension, hypercholesterolemia, cardiovascular disease, renal function disorder, thyroid disorder, obesity, depression anxiety, osteoporosis, liver disorder, autoimmune disease, dementia, Alzheimer's disease.

Abbreviations and acronyms as used herein have respective meanings as follows:

| | Abbreviation/ | |
|---|---|---|
| Category | Acronym | Full Name/Description |
| Solvents | ACN | Acetonitrile |
| | t-BuOH | Tert-butyl alcohol |
| | CPME | Cyclopentyl methyl ether |
| | DCM | Dichloromethane |
| | DMAc | N,N-Dimethylacetamide |
| | DMF | Dimethylformamide |
| | DMSO | Dimethylsulfoxide |
| | EtOH | Ethanol |
| | EtOAc | Ethyl acetate |
| | IPA | Isopropanol |
| | MEK | Methyl ethyl ketone |
| | MeOH | Methanol |
| | MIBK | Methyl isobutyl ketone |
| | MTBE | Methyl-tert-butyl ether |
| | TFE | Trifluoroethanol |
| Analytical | DSC | Differential scanning calorimetry |
| Techniques | DVS | Dynamic vapor sorption |
| | ^{1H}NMR | Proton nuclear magnetic |
| | | resonance spectroscopy |
| | SCXRD | Single crystal x-ray diffraction |
| | TGA | Thermogravimetric analysis |
| | XRPD | X-ray powder diffraction |

### Characterization of Compounds and Solid State Forms

In one embodiment, the present invention provides solid state forms of Compound 1. In one embodiment, the crystalline forms are characterized by the interlattice plane intervals determined by a X-ray powder diffraction (XRPD) diffractogram. The diffractogram is typically represented by a diagram plotting the intensity of the peaks versus the location of the peaks, i.e., diffraction angle 2Θ (two-theta) in degrees. The characteristic peaks of a given compound can be selected according to the peak locations and their relative intensity to distinguish compounds and crystalline structures from others. Amorphous solid state forms were also characterized by XRPD. Amorphous solid state forms exhibit an absence of interlattice plane intervals.

Both crystalline and amorphous solid state forms were identified for Compound 1. Amorphous solid state forms as described herein are specifically denoted as such. For example, the language “solid state form of Compound 1” is meant to describe a crystalline form of Compound 1 unless specified as an amorphous solid state form.

Those skilled in the art recognize that the measurements of the XRPD peak locations and/or intensity for a given crystalline form of the same compound will vary within a margin of error. The values of degree 2Θ allow appropriate error margins. Typically, the error margins are represented by “±”. For example, the degree 2Θ of “8.716±0.3” denotes a range from 9.016 to 8.416. Depending on the sample preparation techniques, the calibration techniques applied to the instruments, human operational variation, and etc., those skilled in the art recognize that the margin of error for a XRPD can be ±0.5; ±0.4; ±0.3; ±0.2; ±0.1; ±0.05; or less. Additional details of the methods and equipment used for the XRD analysis are described in the Examples section.

In one embodiment, the crystalline forms are characterized by Differential Scanning calorimetry (DSC) and Thermogravimetric Analysis (TGA). The DSC thermogram is typically expressed by a diagram plotting the normalized heat flow in units of Watts/gram (“W/g”) versus the measured sample temperature in degree C. The DSC thermogram is generally evaluated for extrapolated onset and end (outset) temperatures, peak temperature, and heat of fusion. The single maximum value of a DSV thermogram is often used as the characteristic peak to distinguish one crystalline form from another crystalline form. The TGA thermogram is typically expressed by a diagram plotting the weight loss percentage (%) versus the measured sample temperature in degree C. In the figures disclosed herein, DSC and TGA thermograms have been plotted sharing an X axis (temperature), but have distinct Y axes of weight % and heat flow corresponding respectively to TGA and DSC measurements.

Those skilled in the art recognize that the measurements of the DSC and TGA thermograms for a given crystalline form of the same compound will vary within a margin of error. The values of a single maximum value, expressed in degree C., allow appropriate error margins. Typically, the error margins are represented by “±”. For example, the single maximum value of “53.1° C.±10.0” denotes a range from 53.1° C.±10.0, i.e., 63.1° C., to about 53.1° C.-10.0, i.e., 43.1° C. Depending on the sample preparation techniques, crystallization conditions, calibration techniques applied to the instruments, human operational variations, and etc., those skilled in the art recognize that the appropriate margin of error for a single maximum value can be ±10.0; ±7.5; ±5.0; ±2.5; ±2; ±1.5; ±1; ±0.5; or less for any of the powder diffraction reflections described herein.

### EMBODIMENTS

Embodiment 1 of this disclosure relates to a solid form of (E)-1-(4-(4-((4-([1,2,4]triazolo[1,5-a]pyridin-7-yloxy)-3-methylphenyl)amino)pyrrolo[2,1-f][1,2,4]triazin-5-yl)piperidin-1-yl)-3-(dimethylamino)prop-2-en-1-one, shown as Compound 1:

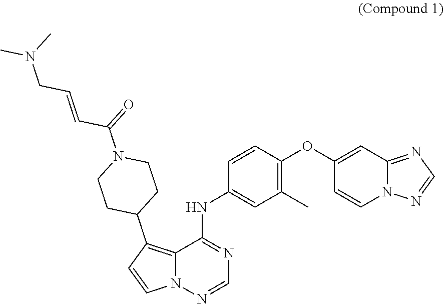 [SMILES: Cc1cc(Nc2ncnn3ccc(C4CCN(C(=O)/C=C/CN(C)C)CC4)c23)ccc1Oc1ccn2ncnc2c1]

wherein the solid form is crystalline.

Embodiment 2 of this disclosure relates to the solid form of Embodiment 1, wherein the solid form exhibits an X-ray powder diffraction (XRPD) reflection at a 2-theta value of 15.3°±0.3.

Embodiment 3 of this disclosure relates to the solid form of Embodiment 2, wherein the solid form exhibits an X-ray powder diffraction (XRPD) reflection at a 2-theta value of 20.8°±0.3.

Embodiment 4 of this disclosure relates to the solid form of Embodiment 2 or 3, wherein the solid form exhibits an X-ray powder diffraction (XRPD) reflection at a 2-theta value of 19.0°±0.3.

Embodiment 5 of this disclosure relates to the solid form of Embodiments 2 to 4, wherein the solid form exhibits an X-ray powder diffraction (XRPD) reflection at a 2-theta value of 12.4°±0.3 and 13.4°±0.3.

Embodiment 6 of this disclosure relates to the solid form of Embodiments 2 to 5, wherein the solid form exhibits an X-ray powder diffraction (XRPD) reflection at a 2-theta value of 5.6°±0.3, 6.7°±0.3, and 8.2°±0.3.

Embodiment 7 of this disclosure relates to the solid form of Embodiments 2 to 6, wherein the solid form exhibits an X-ray powder diffraction (XRPD) reflection at a 2-theta value of 16.4°±0.3, 16.5°±0.3, and 19.4°±0.3.

Embodiment 8 of this disclosure relates to the solid form of Embodiments 2 to 7, wherein the solid form exhibits an X-ray powder diffraction (XRPD) reflection at a 2-theta value of 17.5°±0.3 and 22.8°±0.3.

Embodiment 9 of this disclosure relates to the solid form of Embodiment 1, wherein the solid form exhibits an X-ray powder diffraction (XRPD) reflection at a 2-theta value of 6.7°±0.3.

Embodiment 10 of this disclosure relates to the solid form of Embodiment 9, wherein the solid form exhibits an X-ray powder diffraction (XRPD) reflection at a 2-theta value of 5.6°±0.3 and 8.2°±0.3.

Embodiment 11 of this disclosure relates to the solid form of Embodiment 9 or 10, wherein the solid form exhibits an X-ray powder diffraction (XRPD) reflection at a 2-theta value of 15.3°±0.3, 20.8°±0.3, and 19.0°±0.3.

Embodiment 12 of this disclosure relates to the solid form of Embodiments 9 to 11, wherein the solid form exhibits an X-ray powder diffraction (XRPD) reflection at a 2-theta value of 12.4°±0.3 and 13.4°±0.3.

Embodiment 13 of this disclosure relates to the solid form of Embodiments 9 to 12, wherein the solid form exhibits an X-ray powder diffraction (XRPD) reflection at a 2-theta value of 17.5°±0.3 and 22.8°±0.3.

Embodiment 14 of this disclosure relates to the solid form of Embodiments 9 to 13, wherein the solid form exhibits an X-ray powder diffraction (XRPD) reflection at a 2-theta value of 16.4°±0.3, 16.5°±0.3, and 19.4°±0.3.

Embodiment 15 of this disclosure relates to the solid form of Embodiment 1, wherein the solid form exhibits at least one X-ray powder diffraction (XRPD) reflections at a 2-theta value of 5.6°±0.3, 6.7°±0.3, 8.2°±0.3, 12.4°±0.3, 13.4°±0.3, 15.3°±0.3, 16.4°±0.3, 16.5°±0.3, 17.5°±0.3, 19.0°±0.3, 19.4°±0.3, 20.8°±0.3, and 22.8°±0.3.

Embodiment 16 of this disclosure relates to the solid form of Embodiment 15, wherein the solid form exhibits at least two X-ray powder diffraction (XRPD) reflections at a 2-theta value of 5.6°±0.3, 6.7°±0.3, 8.2°±0.3, 12.4°±0.3, 13.4°±0.3, 15.3°±0.3, 16.4°±0.3, 16.5°±0.3, 17.5°±0.3, 19.0°±0.3, 19.4°±0.3, 20.8°±0.3, and 22.8°±0.3.

Embodiment 17 of this disclosure relates to the solid form of Embodiment 16, wherein the solid form exhibits at least three X-ray powder diffraction (XRPD) reflections at a 2-theta value of 5.6°±0.3, 6.7°±0.3, 8.2°±0.3, 12.4°±0.3, 13.4°±0.3, 15.3°±0.3, 16.4°±0.3, 16.5°±0.3, 17.5°±0.3, 19.0°±0.3, 19.4°±0.3, 20.8°±0.3, and 22.8°±0.3.

Embodiment 18 of this disclosure relates to the solid form of Embodiment 17, wherein the solid form exhibits at least four X-ray powder diffraction (XRPD) reflections at a 2-theta value of 5.6°±0.3, 6.7°±0.3, 8.2°±0.3, 12.4°±0.3, 13.4°±0.3, 15.3°±0.3, 16.4°±0.3, 16.5°±0.3, 17.5°±0.3, 19.0°±0.3, 19.4°±0.3, 20.8°±0.3, and 22.8°±0.3.

Embodiment 19 of this disclosure relates to the solid form of Embodiment 18, wherein the solid form exhibits at least five X-ray powder diffraction (XRPD) reflections at a 2-theta value of 5.6°±0.3, 6.7°±0.3, 8.2°±0.3, 12.4°±0.3, 13.4°±0.3, 15.3°±0.3, 16.4°±0.3, 16.5°±0.3, 17.5°±0.3, 19.0°±0.3, 19.4°±0.3, 20.8°±0.3, and 22.8°±0.3.

Embodiment 20 of this disclosure relates to the solid form of Embodiment 19, wherein the solid form exhibits at least six X-ray powder diffraction (XRPD) reflections at a 2-theta value of 5.6°±0.3, 6.7°±0.3, 8.2°±0.3, 12.4°±0.3, 13.4°±0.3, 15.3°±0.3, 16.4°±0.3, 16.5°±0.3, 17.5°±0.3, 19.0°±0.3, 19.4°±0.3, 20.8°±0.3, and 22.8°±0.3.

Embodiment 21 of this disclosure relates to the solid form of Embodiment 1, wherein the solid form exhibits the X-ray powder diffraction (XRPD) pattern as shown in FIG. 1A.

Embodiment 22 of this disclosure relates to the solid form of Embodiments 1 to 20, wherein the X-ray powder diffraction (XRPD) was collected using a Cu-Kα radiation source.

Embodiment 23 of this disclosure relates to the solid form of Embodiments 1 to 22, wherein the solid form exhibits a differential scanning calorimetry thermograph comprising an endothermic peak at 171.3±5.0° C.

Embodiment 24 of this disclosure relates to the solid form of Embodiments 1 to 23, wherein the solid form exhibits a differential scanning calorimetry thermograph as shown in FIG. 2.

Embodiment 25 of this disclosure relates to the solid form of Embodiments 1 to 24, wherein the solid form exhibits less than 1.0%±0.3 weight loss up to 214° C.±10.0 as determined by thermogravimetric analysis.

Embodiment 26 of this disclosure relates to the solid form of Embodiment 1 to 25, wherein the solid form is substantially free of other crystalline or amorphous forms.

Embodiment 27 of this disclosure relates to the solid form of Embodiment 26, wherein the amount of other crystalline or amorphous forms is 5% (w/w) or less.

Embodiment 28 of this disclosure relates to the solid form of Embodiment 26, wherein the amount of other crystalline or amorphous forms is 2% (w/w) or less.

Embodiment 29 of this disclosure relates to the solid form of Embodiments 1 to 28, wherein the solid form is substantially free of impurities.

Embodiment 30 of this disclosure relates to the solid form of Embodiment 29, wherein the amount of impurities is 2% or less.

Embodiment 31 of this disclosure relates to a pharmaceutical composition comprising any one of the solid forms of Embodiments 1 to 30, and one or more pharmaceutically acceptable excipients or carriers

Embodiment 32 of this disclosure relates to a method of treating cancer in a subject, the method comprising administering to the subject an effective amount of the solid form of any one of Embodiments 1 to 30, or the pharmaceutical composition of Embodiment 31.

Embodiment 33 of this disclosure replates to the method of Embodiment 32, wherein the cancer is HER2 mediated.

Embodiment 34 of this disclosure replates to the method of Embodiment 32 or 33, wherein the caner is brain cancer, colorectal cancer, breast cancer, bladder cancer, or non-small cell lung cancer.

Embodiment 35 of this disclosure replates to the method of Embodiment 34, wherein the cancer is colorectal cancer, breast cancer, bladder cancer, or non-small cell lung cancer.

Embodiment 36 of this disclosure replates to the method of Embodiments 32 to 35, wherein the method further comprises administering an additional therapeutic agent.

Embodiment 37 of this disclosure replates to the method of Embodiment 36, wherein the additional therapeutic agent is a monoclonal antibody.

Embodiment 38 of this disclosure replates to the method of Embodiment 37, wherein the monoclonal antibody is pertuzumab, margetuximab, or trastuzumab.

Embodiment 39 of this disclosure replates to the method of Embodiment 38, wherein the monoclonal antibody is trastuzumab.

Embodiment 40 of this disclosure replates to the method of Embodiment 36, wherein the additional therapeutic agent is a monoclonal antibody-drug conjugate.

Embodiment 41 of this disclosure replates to the method of Embodiment 40, wherein the monoclonal antibody-drug conjugate is ado-trastuzumab emtansine or trastuzumab deruxtecan.

### EXAMPLES

The following illustrative examples are representative of embodiments of the stimulation, systems, and methods described herein and are not meant to be limiting in any way

### Example 1. Synthesis of Crystalline Compound 1

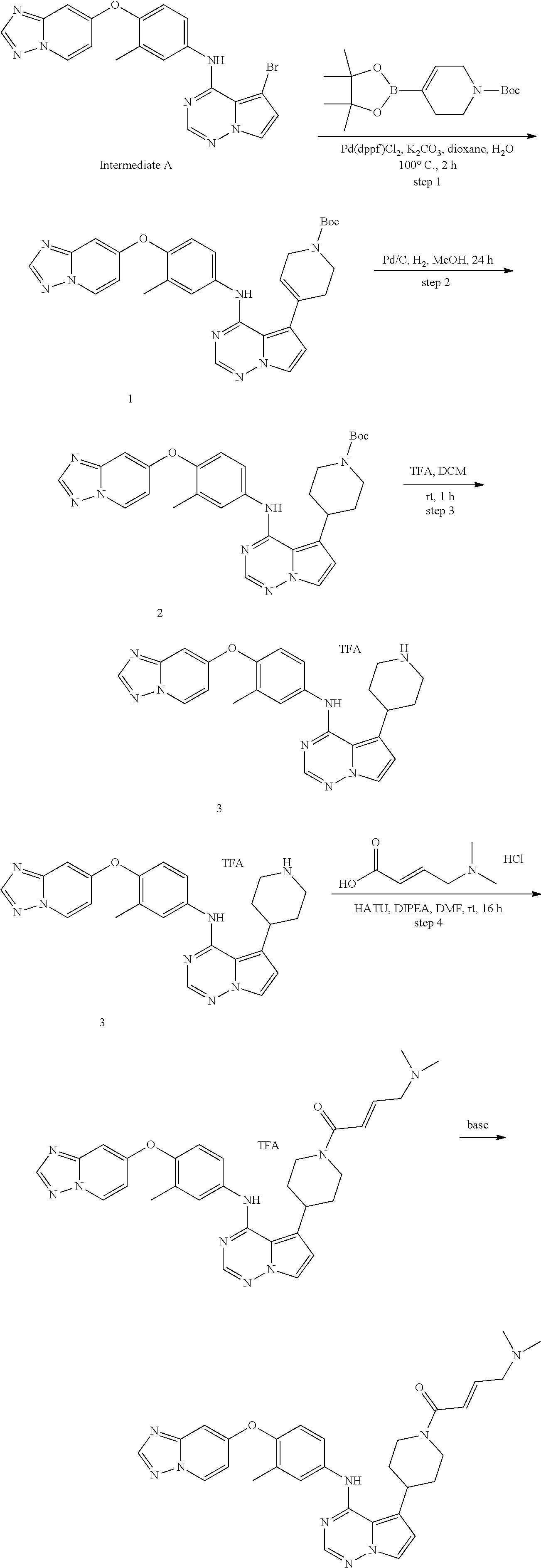

### Synthesis of Intermediate A

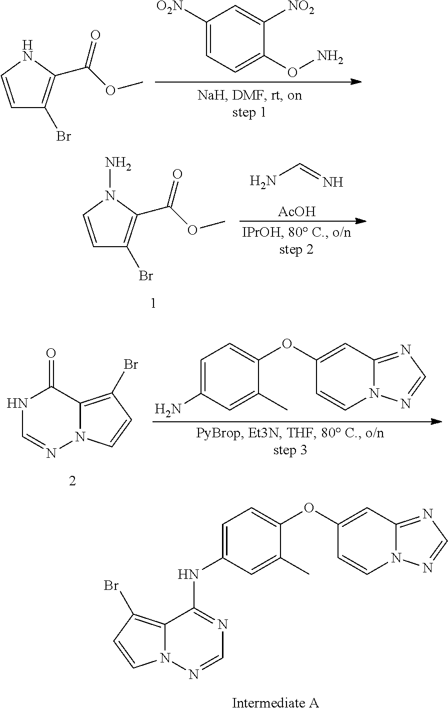

Step 1. Methyl 1-amino-3-bromo-1H-pyrrole-2-carboxylate

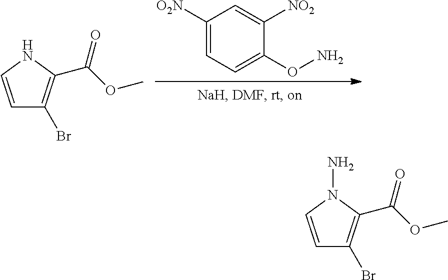

A solution of methyl 3-bromo-1H-pyrrole-2-carboxylate (25 g, 122.53 mmol) in DMF (200 mL) and THF (1000 mL) was treated with NaH (60% in mineral oil, 6.37 g, 159.25 mmol) for 1 hour at 0° C. followed by the addition of O-(2,4-dinitrophenyl) hydroxylamine (29.28 g, 147.04 mmol) at 0° C. and the mixture was stirred for 16 hours at room temperature. The reaction was quenched by the addition of saturated ammonium chloride aqueous solution (500 ml) at 0° C. The resulting mixture was diluted with water (1 L) and extracted with ethyl acetate (1.5 L×2). The combined organic layers were washed with brine (1.5 L×2), dried over anhydrous sodium sulfate, concentrated under reduced pressure. The residue was purified by silica gel column chromatography (0-20% ethyl acetate in hexanes) to provide methyl 1-amino-3-bromo-1H-pyrrole-2-carboxylate (22 g, 81.97%). LCMS (ESI-MS) m/z=219.0 [M+H]⁺

Step 2. 5-bromopyrrolo[2,1-f][1,2,4]triazin-4(3H)-one

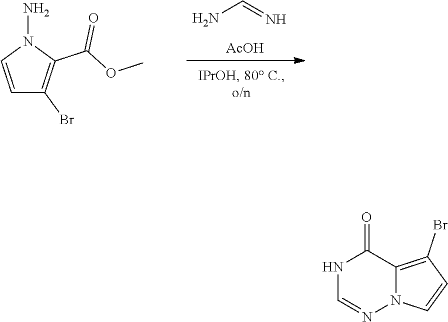

To a stirred solution of methyl 1-amino-3-bromo-1H-pyrrole-2-carboxylate (22 g, 100.43 mmol) in iPrOH (150 mL) was added formimidamide acetate (20.91 g, 200.87 mmol). The mixture was stirred at 80° C. overnight. The resulting mixture was diluted with water (300 ml). The precipitated solids were collected by filtration and washed with water (100 ml×3) and petroleum ether (200 ml) to afford 5-bromopyrrolo[2,1-f][1,2,4]triazin-4 (3H)-one (13.2 g crude). LCMS (ESI-MS) m/z=214.0 [M+H]⁺.

Step 3. N-(4-([1,2,4]triazolo[1,5-a]pyridin-7-yloxy)-3-methylphenyl)-5-bromopyrrolo[2,1-f][1,2,4]triazin-4-amine

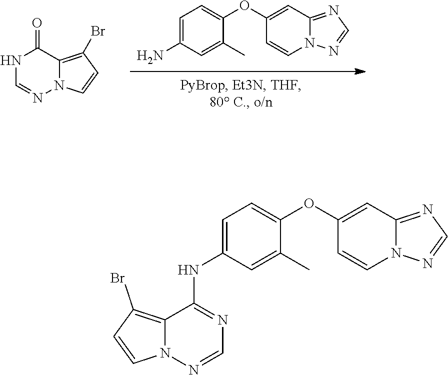 [SMILES: O=c1[nH]cnn2ccc(Br)c12 ; Cc1cc(N)ccc1Oc1ccn2ncnc2c1 ; Cc1cc(Nc2ncnn3ccc(Br)c23)ccc1Oc1ccn2ncnc2c1]

A solution of 5-bromopyrrolo[2,1-f][1,2,4]triazin-4(3H)-one (13.2 g, 61.97 mmol), 4-([1,2,4]triazolo[1,5-a]pyridin-7-yloxy)-3-methylaniline (14.87 g, 61.97 mmol), PyBrop (43.31 g, 92.95 mmol) and Et₃N (18.81 g, 185.91 mmol) in THF (300 mL) was stirred overnight at 80° C. The resulting mixture was purified by silica gel column chromatography (0-80% ethyl acetate in hexanes) to afford the title compound N-(4-([1,2,4]triazolo[1,5-a]pyridin-7-yloxy)-3-methylphenyl)-5-bromopyrrolo[2,1-f][1,2,4]triazin-4-amine, intermediate A (12 g, 27.33%). LCMS (ESI-MS) m/z=436.0 [M+H]⁺.

Step 1. tert-butyl 4-(4-((4-([1,2,4]triazolo[1,5-a]pyridin-7-yloxy)-3-methylphenyl)amino)pyrrolo[2,1-f][1,2,4]triazin-5-yl)-3,6- dihydropyridine-1(2H)-carboxylate

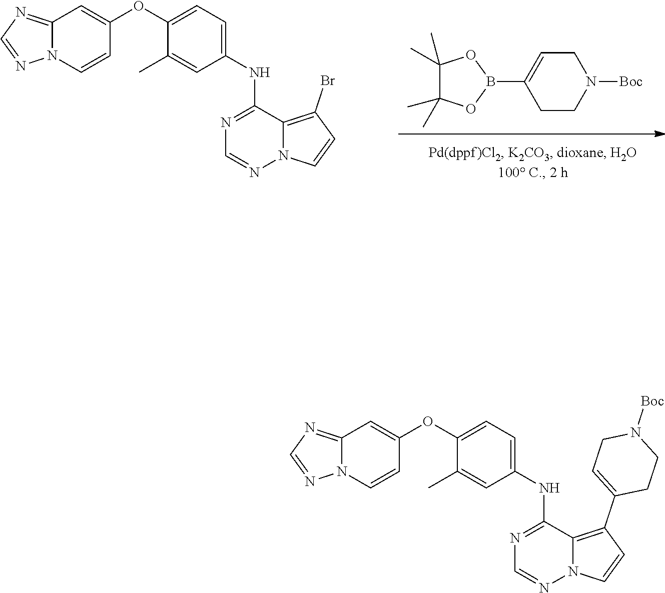 [SMILES: Cc1cc(Nc2ncnn3ccc(Br)c23)ccc1Oc1ccn2ncnc2c1 ; CC(C)(C)OC(=O)N1CC=C(B2OC(C)(C)C(C)(C)O2)CC1 ; Cc1cc(Nc2ncnn3ccc(C4=CCN(C(=O)OC(C)(C)C)CC4)c23)ccc1Oc1ccn2ncnc2c1]

A solution of N-(4-([1,2,4]triazolo[1,5-a]pyridin-7-yloxy)-3-methylphenyl)-5-bromopyrrolo[2,1-f][1,2,4]triazin-4-amine (200 mg, 0.46 mmol), tert-butyl 4-(4,4,5,5-tetramethyl-1,3,2-dioxaborolan-2-yl)-3,6-dihydropyridine-1(2H)-carboxylate (141.75 mg, 0.46 mmol), Pd(dppf)Cl₂ (37.34 mg, 0.05 mmol) and K₂CO₃ (126.71 mg, 0.92 mmol) in dioxane (4 mL) and H₂O (1.2 mL) was stirred for 2 hours at 100° C. under nitrogen atmosphere. The resulting mixture was concentrated under vacuum to afford the crude product. The crude product was purified by Prep-TLC (petroleum ether/ethyl acetate 1:10) to afford tert-butyl 4-(4-((4-([1,2,4]triazolo[1,5-a]pyridin-7-yloxy)-3-methylphenyl)amino)pyrrolo[2,1-f][1,2,4]triazin-5-yl)-3,6-dihydropyridine-1(2H)-carboxylate (220 mg, purity=96.8%). LCMS (ESI-MS) m/z=539.2 [M+H]⁺.

Step 2. Tert-butyl 4-(4-((4-([1,2,4]triazolo[1,5-a]pyridin-7-yloxy)-3-methylphenyl)amino)pyrrolo[2,1-f][1,2,4]triazin-5-yl)piperidine-1- carboxylate

A solution of 4-(4-((4-([1,2,4]triazolo[1,5-a]pyridin-7-yloxy)-3-methylphenyl)amino)pyrrolo[2,1-f][1,2,4]triazin-5-yl)-3,6-dihydropyridine-1(2H)-carboxylate (200 mg, 0.37 mmol) and Pd/C (395.16 mg, 3.71 mmol) in MeOH was stirred overnight at room temperature under hydrogen atmosphere. The mixture was filtered off and the filtrate was concentrated under vacuum to afford crude product. The crude product was used in the next step directly without further purification. LCMS (ESI-MS) m/z=541.3 [M+H]⁺.

Step 3. N-(4-([1,2,4]triazolo[1,5-a]pyridin-7-yloxy)-3-methylphenyl)-5-(piperidin-4-yl)pyrrolo[2,1-f][1,2,4]triazin-4-amine

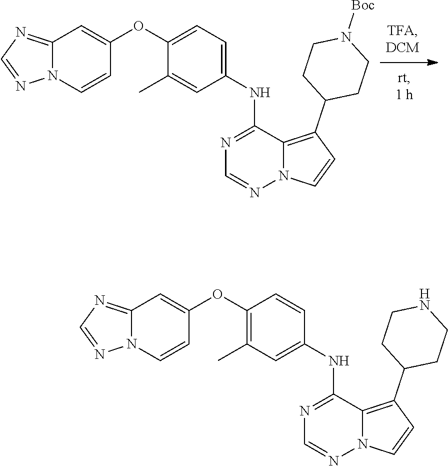 [SMILES: Cc1cc(Nc2ncnn3ccc(C4CCN(C(=O)OC(C)(C)C)CC4)c23)ccc1Oc1ccn2ncnc2c1 ; Cc1cc(Nc2ncnn3ccc(C4CCNCC4)c23)ccc1Oc1ccn2ncnc2c1]

A solution of tert-butyl 4-(4-((4-([1,2,4]triazolo[1,5-a]pyridin-7-yloxy)-3-methylphenyl)amino)pyrrolo[2,1-f][1,2,4]triazin-5-yl)piperidine-1- carboxylate (140 mg, 0.26 mmol) in TFA (2 mL) was stirred for 1 hour at room temperature. The resulting mixture was concentrated under vacuum to afford the crude product. The crude product was used in the next step directly without further purification. LCMS (ESI-MS) m/z=441.2 [M+H]⁺.

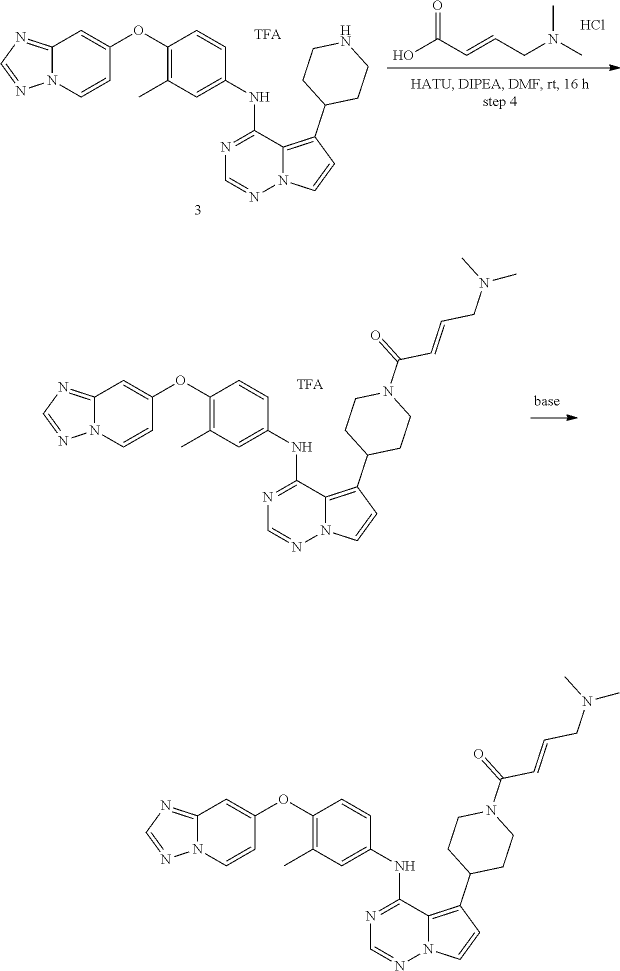

(E)-1-(4-(4-((4-([1,2,4]triazolo[1,5-a]pyridin-7-yloxy)-3-methylphenyl)amino)pyrrolo[2,1-f][1,2,4]triazin-5-yl)piperidin-1-yl)-4-(dimethylamino)but-2-en-1-one
Step 4. To a 20 mL scintillation vial with Teflon-coated stir bar was added N-(4-([1,2,4]triazolo[1,5-a]pyridin-7-yloxy)-3-methylphenyl)-5-(piperidin-4-yl)pyrrolo[2,1-f][1,2,4]triazin-4-amine (TFA salt, 50.0 mg, 1 Eq, 90.2 μmol), (E)-4-(dimethylamino)but-2-enoic acid hydrochloride (22.4 mg, 1.5 Eq, 135 μmol), and HATU (51.4 mg, 1.5 Eq, 135 μmol). The vial was then capped with a rubber septum and evacuated and refilled with N₂ (3×). Then DMF (3.0 mL) and Diisopropylethylamine (46.6 mg, 62.3 μL, 4 Eq, 361 μmol) were added via syringe and the reaction was stirred overnight at rt. The resulting mixture was filtered then purified by preparative reverse phase HPLC (acetonitrile/water gradient with 0.1% TFA) to afford 2,2,2-trifluoroacetaldehyde-(E)-1-(4-(4-((4-([1,2,4]triazolo[1,5-a]pyridin-7-yloxy)-3-methylphenyl)amino)pyrrolo[2,1-f][1,2,4]triazin-5-yl)piperidin-1-yl)-4-(dimethylamino)but-2-en-1-one (2.0 mg, 3.3% yield. LCMS (ESI) [M+H]⁺=552.3. TFA was subsequently removed by exposure to base to yield Compound 1. Conditions for the formation of Form A can be seen below in Table 1.

TABLE 1

| Generation | Generation | Analytical | |
|---|---|---|---|
| Conditions | Conditions | Technique | Results |
| Form A | 0% RH | XRPD | XRPD pattern indexed, single crystalline |
| | | | phase |
| | | KF | 0.22 wt % (0.07 mol/mol H₂O), difference in |
| | | | unit cell volume is ~1 mol/mol H₂O:API |
| | 75% RH | XRPD | XRPD pattern indexed, single crystalline |
| | | | phase |
| | | KF | 2.34 wt % (0.73 mol/mol H₂O), difference in |
| | | | unit cell volume is ~1 mol/mol H₂O:API |
| | ACN slurry | XRPD | XRPD pattern indexed, single crystalline |
| | | | phase |
| | | ¹H NMR | Consistent w/chemical structure |
| | | | Dioxane (less than ~5 mmol/mol), based on |
| | | | singlet at 3.57 ppm |
| | | | MTBE (less than ~5 mmol/mol), based on |
| | | | singlets at 3.08 and 1.11 ppm |
| | | | ACN (~0.1 mol/mol), based on singlet at |
| | | | 2.07 ppm |
| | | | Trace amounts of impurities possible |
| | 40:60 acetone/water | XRPD | XRPD pattern indexed, single crystalline |
| | slurry | | phase |
| | 20:80 DMAc/MTBE | XRPD | XRPD pattern indexed, single crystalline |
| | slurry | | phase |
| | acetone | ¹H NMR | Consistent w/chemical structure |
| | fast evaporation | | Acetone (~0.5 mol/mol), based on singlet at |
| | | | 2.09 ppm |
| | | | Dioxane (less than ~0.01 mol/mol), based on |
| | | | singlet at 3.57 ppm |
| | | | Trace amounts of impurities, increased |
| | | | compared to as-received |
| | chloroform/MTBE | ¹H NMR | Consistent w/chemical structure |
| | vapor diffusion | | MTBE (~0.06 mol/mol), based on singlets at |
| | | | 3.08 and 1.11 ppm |
| | | | Chloroform (~0.04 mol/mol), based o singlet |
| | | | at 8.32 ppm |
| | | | Trace amounts of impurities likely |
| X-ray | DCM rotary | XRPD | Diffuse scatter, no peaks |
| amorphous | evaporation | ¹H NMR | In progress |
| | | TMDSC | In progress |

### Example 2. Thermal Analysis of Crystalline Compound 1

Cycling DSC was completed in order to ideally separate thermal events leading to asymmetrical endotherm. The DSC cycles included heating the material to 190° C., holding the material at 190° C. for 5 minutes, quenching the material to −30° C., reheating the sample to 130° C. at a rate of 10° C./min, and continuing to slowly heat the material to 190° C. at a rate of 2° C./min.

The initial heating of material to 190° C. was done in order to heat the sample past the potential melt that was previously observed in the TGA/DSC thermogram of the as-received material. Doing this would ensure the removal of volatiles observed in the as-received TGA and the completeness of the melt, assuming that this thermal event is indeed due to a melt.

By DSC, a broad endotherm with a peak maximum of 55.2° C. followed by a sharp endotherm with an onset at 169.7° C. was observed. The second endotherm is strong but asymmetric, suggesting overlapping events as indicated by the corresponding loss in the TGA at the same temperature.

The material was held at 190° C. for 5 minutes, this was done in order to ensure that all of the material was melted. No events were observed. The sample was then quenched to −30° C. as an attempt at the in-situ generation of amorphous material. A subsequent reheating of the material was conducted up to 130° C. at a rate of 10° C./min in order to observe any thermal events associated with the potentially amorphous material generated via the quench.

A step change, including a minor endotherm-like event associated was observed with a midpoint at 85.6° C. and a ΔC_{P} of 0.4 J/gK, indicating that it may represent the glass transition of the amorphous material.

Finally, the heating of the sample continued to 190° C. at a rate of 2° C./min in order to catch any remaining thermal events including crystallization. No events were observed.

In conclusion, the experiment successfully generated amorphous material as indicated by the step change likely representing the glass transition of the material upon reheating. No crystallization of the amorphous material was observed during the reheating, even with the use of a slower heating rate.

Thermal characterization by TGA showed a 0.6% weight loss from 42 to 97° C. followed by a secondary 0.3% weight loss from 131 to 189° C., these were found to be equivalent to a loss of 0.19 and <0.1 mol/mol of water respectively but could also be due to residual organic solvents. What is likely a decomposition onset of the material was observed at 214° C.

### Example 3. Analytical Data of Crystalline Compound 1 Form A

The characterization data (thermographic, XRPD, and DVS) suggests that Form A may be a variable hydrate. Variable hydrates are crystalline material that may have variable water content over an extended relative humidity range, yet with no phase change. Such solid-state forms can be useful in pharmaceutical formulations, as differences in humidity have less of an effect on stability.

### XRPD

The XRPD data was collected with Cu-Kα radiation. The XRPD pattern was successfully indexed and indicative of a single crystalline phase compound.

| | Compound 1 |
|---|---|
| Bravais Type | Primitive |
| | Orthorhombic |
| a [Å] | 6.310 |
| b [Å] | 14.590 |
| c [Å] | 31.638 |
| α [deg] | 90 |
| β [deg] | 90 |
| γ [deg] | 90 |
| Volume [Å³/cell] | 2,912.7 |
| Chiral Contents? | Not Specified |
| Extinction Symbol | P 2₁ 2₁ 2₁ |
| Space Groups(s) | P2₁2₁2₁ (19) |
| Source | Manual Input |

Table 2 below reports the numerical values of the XRPD of Compound 1 Form A shown in FIG. 1A and FIG. 1B.

TABLE 2

| | d space | Intensity |
|---|---|---|
| °2θ | (Å) | (%) |
| 5.56 | 15.882 | 31 |
| 6.65 | 13.281 | 34 |
| 8.22 | 10.748 | 24 |
| 10.33 | 8.557 | 7 |
| 11.16 | 7.922 | 5 |
| 12.44 | 7.110 | 36 |
| 12.71 | 6.959 | 12 |
| 13.35 | 6.627 | 37 |
| 14.31 | 6.184 | 5 |
| 14.75 | 6.001 | 11 |
| 15.27 | 5.798 | 100 |
| 16.36 | 5.414 | 26 |
| 16.51 | 5.365 | 29 |
| 16.79 | 5.276 | 14 |
| 17.45 | 5.078 | 29 |
| 17.87 | 4.960 | 6 |
| 18.57 | 4.774 | 18 |
| 18.97 | 4.674 | 63 |
| 19.41 | 4.569 | 37 |
| 20.10 | 4.414 | 24 |
| 20.41 | 4.348 | 9 |
| 20.77 | 4.273 | 66 |
| 21.44 | 4.141 | 20 |
| 21.73 | 4.087 | 14 |
| 21.95 | 4.046 | 14 |
| 22.79 | 3.899 | 35 |
| 23.10 | 3.847 | 14 |
| 23.32 | 3.811 | 16 |
| 23.77 | 3.740 | 8 |
| 24.21 | 3.673 | 24 |
| 24.57 | 3.620 | 21 |
| 25.01 | 3.558 | 19 |
| 25.12 | 3.542 | 19 |
| 25.63 | 3.473 | 16 |
| 26.62 | 3.346 | 5 |
| 27.29 | 3.265 | 17 |
| 28.22 | 3.160 | 9 |
| 28.69 | 3.109 | 9 |
| 29.06 | 3.070 | 10 |
| 29.71 | 3.005 | 9 |
| 30.47 | 2.931 | 6 |
| 31.15 | 2.869 | 4 |
| 31.62 | 2.827 | 9 |
| 32.27 | 2.772 | 5 |
| 33.04 | 2.709 | 3 |
| 33.70 | 2.658 | 5 |
| 33.97 | 2.637 | 7 |
| 34.85 | 2.572 | 5 |
| 35.38 | 2.535 | 3 |
| 36.41 | 2.465 | 3 |
| 36.92 | 2.433 | 7 |

XRPD patterns of various samples of Compound 1 Form A exhibited peaks shifts of ±0.3 under various conditions. Illustration of the shifts is provided in FIG. 5A and FIG. 5B. The figures show stacked XRPD diffractograms under the following conditions: stored at 0% relative humidity; slurried in DMAC/MTBE, as obtained from the procedure described in Example 1; slurried in acetonitrile; stored at 75% relative humidity; slurried in 40:60 acetone/water slurry (a_{w} 0.92). Unit cell volumes contract in the absence of water and expand the presence of water, as shown in Table 3 below.

TABLE 3

| | Unit cell volume from XRPD |
|---|---|
| Generation Conditions | indexing, Å³/cell |
| 0% RH | 2873.7 |
| DMAc/MTBE | 2885.8 |
| As-synthesized | 2912.7 |
| ACN slurry | 2933.9 |
| 75% RH | 2933.6 |
| 40:60 acetone/ | 2956.5 |
| water slurry (a_{w} 0.92) | |

### DVS

By DVS, a weight gain of 2.8 weight %, consistent with a gain of 0.9 mol water, was observed from 6% to 95% RH. A complete desorption was observed from 95% to 6% RH. No significant hysteresis was observed. The results of the experiments are shown in Table 4 below. The resulting crystalline material was analyzed by XRPD and shown to the Form A.

TABLE 4

| | | | Sorp | | Desorp | |
|---|---|---|---|---|---|---|
| | Target | Sample | Mass | Sample | Mass | |
| | RH | RH | Change | RH | Change | |
| | (%) | (%) | (%) | (%) | (%) | Hysteresis |
| Cycle 1 | 5.0 | 5.5 | 0.004 | 5.8 | −0.017 | |
| | 15.0 | 15.5 | 0.277 | 15.3 | 0.271 | −0.006 |
| | 25.0 | 24.6 | 0.609 | 25.0 | 0.648 | 0.039 |
| | 35.0 | 34.3 | 1.034 | 34.8 | 1.109 | 0.075 |
| | 45.0 | 44.4 | 1.470 | 44.9 | 1.573 | 0.104 |
| | 55.0 | 54.3 | 1.811 | 54.8 | 1.934 | 0.123 |
| | 65.0 | 64.4 | 2.087 | 64.9 | 2.199 | 0.112 |
| | 75.0 | 74.5 | 2.315 | 75.1 | 2.431 | 0.116 |
| | 85.0 | 85.1 | 2.559 | 86.2 | 2.656 | 0.096 |
| | 95.0 | 95.4 | 2.849 | 95.4 | 2.849 | |

### Solubility

Solubilities were estimated by solvent aliquot addition at ambient temperature and reported to the nearest whole number; if complete dissolution was achieved by one aliquot addition, the value is reported as “>”; if solids were present, the value is reported as “<”. The actual solubility may be larger than the value calculated because of the use of solvent aliquots that were too large or due to a slow rate of dissolution. Table 5 below shows the solubility of Compound 1 Form A.

TABLE 5

| Solvent System v/v %\| | Solubility (mg/mL) | Observations |
|---|---|---|
| Acetone | 15 | Clear soln. |
| ACN | 7 | Clear soln. |
| chloroform | >100 | Clear soln. |
| 10:90 chloroform/IPA | 27 | Clear soln. |
| cyclohexane | <1 | Solids |
| DCM | >88 | Clear soln. |
| 50:50 DCM/toluene | 50 | Clear soln. |
| 30:70 DCM/toluene | 9 | Clear soln. |
| dioxane | 32 | Clear soln. |
| DMAc | >110 | Clear soln. |
| 50:50 DMAc/toluene | >28 | Clear soln. |
| 30:70 DMAc/MTBE | 29 | Clear soln. |
| EtOAc | 3 | Clear soln. |
| 50:50 EtOAc/IPA | 17 | Clear soln. |
| 50:50 EtOAc/MIBK | 4 | Clear soln. |
| 30:70 EtOH/toluene | >88 | Clear soln. |
| heptane | <2 | Solids |
| IPA | 3 | Clear soln. |
| MeOH | >116 | Clear soln. |
| 20:80 MeOH/EtOAc | >51 | Clear soln. |
| MIBK | 3 | Clear soln. |
| MTBE | <1 | Solids |
| THF | 38 | Clear soln. |
| 70:30 THF/heptane | 10 | Clear soln. |
| 2-MeTHF | 5 | Clear soln. |
| toluene | <1 | Minimal solids |
| water | <1 | Solids |
| 50:50 acetone/water | 24 | Clear soln. |
| 50:50 ACN/water | >72 | Clear soln. |
| 10:90 dioxane/water | <1 | solids |
| 50:50 dioxane/water | 29 | Clear soln. |
| 50:50 DMAc/water | 10 | Clear soln. |
| 20:80 MeOH/water | <1 | solids |
| 50:50 MeOH/water | 4 | Clear soln. |
| 50:50 THF/water | >31 | Clear soln. |
| 35:65 THF/water | 17 | Clear soln. |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A solid form of (E)-1-(4-(4-((4-([1,2,4]triazolo[1,5-a]pyridin-7-yloxy)-3-methylphenyl)amino)pyrrolo[2,1-f][1,2,4]triazin-5-yl)piperidin-1-yl)-3-(dimethylamino)prop-2-en-1-one, shown as Compound 1: 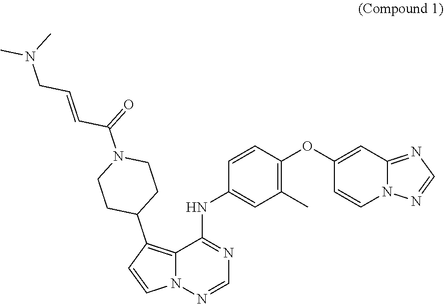 [SMILES: Cc1cc(Nc2ncnn3ccc(C4CCN(C(=O)/C=C/CN(C)C)CC4)c23)ccc1Oc1ccn2ncnc2c1]
wherein the solid form is crystalline.

2. The solid form of claim 1, wherein the solid form exhibits an X-ray powder diffraction (XRPD) reflection at a 2-theta value of 15.3°±0.3.

3. The solid form of claim 2, wherein the solid form exhibits an X-ray powder diffraction (XRPD) reflection at a 2-theta value of 20.8°±0.3.

4. The solid form of claim 2, wherein the solid form exhibits an X-ray powder diffraction (XRPD) reflection at a 2-theta value of 19.0°±0.3.

5. The solid form of claim 2, wherein the solid form exhibits an X-ray powder diffraction (XRPD) reflection at a 2-theta value of 12.4°±0.3 and 13.4°±0.3.

6. The solid form of claim 2, wherein the solid form exhibits an X-ray powder diffraction (XRPD) reflection at a 2-theta value of 5.6°±0.3, 6.7°±0.3, and 8.2°±0.3.

7. The solid form of claim 2, wherein the solid form exhibits an X-ray powder diffraction (XRPD) reflection at a 2-theta value of 16.4°±0.3, 16.5°±0.3, and 19.4°±0.3.

8. The solid form of claim 2, wherein the solid form exhibits an X-ray powder diffraction (XRPD) reflection at a 2-theta value of 17.5°±0.3 and 22.8°±0.3.

9. The solid form of claim 1, wherein the solid form exhibits an X-ray powder diffraction (XRPD) reflection at a 2-theta value of 6.7°±0.3.

10. The solid form of claim 9, wherein the solid form exhibits an X-ray powder diffraction (XRPD) reflection at a 2-theta value of 5.6°±0.3 and 8.2°±0.3.

11. The solid form of claim 9, wherein the solid form exhibits an X-ray powder diffraction (XRPD) reflection at a 2-theta value of 15.3°±0.3, 20.8°±0.3, and 19.0°±0.3.

12. The solid form of claim 9, wherein the solid form exhibits an X-ray powder diffraction (XRPD) reflection at a 2-theta value of 12.4°±0.3 and 13.4°±0.3.

13. The solid form of claim 9, wherein the solid form exhibits an X-ray powder diffraction (XRPD) reflection at a 2-theta value of 17.5°±0.3 and 22.8°±0.3.

14. The solid form of claim 9, wherein the solid form exhibits an X-ray powder diffraction (XRPD) reflection at a 2-theta value of 16.4°±0.3, 16.5°±0.3, and 19.4°±0.3.

15. The solid form of claim 1, wherein the solid form exhibits at least one X-ray powder diffraction (XRPD) reflections at a 2-theta value of 5.6°±0.3, 6.7°±0.3, 8.2°±0.3, 12.4°±0.3, 13.4°±0.3, 15.3°±0.3, 16.4°±0.3, 16.5°±0.3, 17.5°±0.3, 19.0°±0.3, 19.4°±0.3, 20.8°±0.3, and 22.8°±0.3.

16. The solid form of claim 15, wherein the solid form exhibits at least four X-ray powder diffraction (XRPD) reflections at a 2-theta value of 5.6°±0.3, 6.7°±0.3, 8.2°±0.3, 12.4°±0.3, 13.4°±0.3, 15.3°±0.3, 16.4°±0.3, 16.5°±0.3, 17.5°±0.3, 19.0°±0.3, 19.4°±0.3, 20.8°±0.3, and 22.8°±0.3.

17. The solid form of claim 1, wherein the solid form exhibits a differential scanning calorimetry thermograph comprising an endothermic peak at 171.3±5.0° C.

18. The solid form of claim 1, wherein the solid form exhibits less than 1.0%±0.3 weight loss up to 214° C.±10.0 as determined by thermogravimetric analysis.

19. A pharmaceutical composition comprising any one of the solid forms of claim 1, and one or more pharmaceutically acceptable excipients or carriers.

20. A method of treating cancer in a subject, the method comprising administering to the subject an effective amount of the solid form of claim 1.
